(19) United States
(12) Patent Application Publication
(10) Pub. No.: US 2026/0345551 A1
(43) Pub. Date: Nov. 5, 2026

(54) TREATMENT FOR FRAGILE X SYNDROME USING ETHOSUXIMIDE

(71) Applicant: Children's Hospital Medical Center, Cincinnati, OH (US)

(72) Inventors: Craig ERICKSON, Cincinnati, OH (US); Ernest PEDAPATI, Cincinnati, OH (US); Lauren SCHMITT, Cincinnati, OH (US)

(21) Appl. No.: 19/665,310

(22) Filed: May 1, 2026

Related U.S. Application Data
(60) Provisional application No. 63/798,238, filed on May 1, 2025.

(51) Int. Cl. A61K 31/4015; A61K 31/4184; A61K 31/44

(52) U.S. Cl. CPC A61K 31/4015; A61K 31/4184; A61K 31/44

(57) ABSTRACT

Aspects according to the present disclosure include methods for treating a patient with fragile X syndrome by administering an effective amount of an agent that modulates activity of a T-type calcium channel to a patient having fragile X syndrome. Examples of the present disclosure include administering an effective amount of the agent that modulates activity of a T type calcium channel. The agent may be a T-type calcium channel blocker, inhibitor, or antagonist selected from mibefradil, ethosuximide, methsuximide, mesuximide, phensuximide, suvecaltamide, or one or more combinations thereof. Also disclosed are methods of reducing symptoms associated with fragile X syndrome by administering an effective amount of the agent that modulates activity of a T-type calcium channel to a patient having fragile X syndrome. The agent may be a T-type calcium channel blocker, inhibitor, or antagonist selected from mibefradil, ethosuximide, methsuximide, mesuximide, phensuximide, suvecaltamide, or one or more combinations thereof.

### TECHNICAL FIELD

Aspects of the disclosure are directed to treatments of fragile X syndrome. Other aspects of the disclosure are directed to methods of administering ethosuximide to patients having fragile X syndrome. Other aspects of the disclosure are directed to methods for alleviating symptoms of fragile X syndrome in a patient.

### BACKGROUND

Fragile X syndrome is an inherited genetic disorder. Patients with fragile X syndrome can present with a range of behavioral, cognitive, physical, and mental health features. Common manifestations include developmental and learning delays, intellectual disability, anxiety, depression, obsessive compulsive behaviors, attention deficit disorder with or without hyperactivity, reduced IQ, and autism spectrum disorder. Fragile X syndrome results from a mutation of the FMR1 gene on the X chromosome, which regulates the production of the FMRP protein having a role in the development of nerve synapses. It affects males, who have one X chromosome, more than females, who have two X chromosomes. Males typically experience more severe symptoms than females.

Fragile X syndrome currently has no cure. Existing treatments target symptoms of fragile X syndrome. Providers typically attempt to manage symptoms of fragile X syndrome similar to patients having the same symptoms but do not have fragile X syndrome. For example, mood instability may be treated with lithium, ADHD may be treated with methylphenidate or dextroamphetamine, and sleep issues may be treated with melatonin or a hypnotic. Patients may also be treated with therapy.

Ethosuximide is a known agent used to block T-type calcium channels. It has been used to treat absence seizures involving thalamocortical dysrhythmia. Absence seizure patients characteristically exhibit 3 Hz spike wave discharges when measured with an electroencephalogram (“EEG”). Thalamocortical dysrhythmia has been observed in fragile X patients, although fragile X patients exhibit much different patterns on an EEG (such as altered theta/alpha and gamma activity instead of 3-Hz spike-wave discharges). Patients having Parkinson's disease are also known to exhibit thalamocortical dysrhythmia, however treating Parkinson's patients with ethosuximide has been shown to worsen, not improve, symptoms in these patients.

Existing treatments for fragile X syndrome patients are non-specific to the cause of the symptoms, namely fragile X syndrome. Accordingly, there remains a need for treatments for patients with fragile X syndrome that target one or more of the underlying causes of the symptoms associated with fragile X syndrome.

### SUMMARY OF THE DISCLOSURE

It is therefore an object of the present disclosure to provide treatments for patients with fragile X syndrome. It is another object of the present disclosure to provide methods for treating patients with fragile X syndrome that target one or more underlying causes of the symptoms associated with fragile X syndrome.

In one aspect of the present disclosure, a method of treating fragile X syndrome includes administering to a patient having fragile X syndrome an effective amount of an agent that modulates activity of a T-type calcium channel. In an example, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another example, the agent is ethosuximide.

In another aspect of the present disclosure, a method of treating one or more symptoms associated with fragile X syndrome includes administering to a patient having fragile X syndrome an effective amount of an agent that modulates activity of a T-type calcium channel. In an example, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another example, the agent is ethosuximide.

In another aspect of the present disclosure, the agent is administered in an amount effective to raise a peak alpha frequency of the patient. In one aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the present disclosure, the rise in peak alpha frequency causes a decrease in power in a gamma band frequency (greater than 30 Hz) of the patient.

In another aspect of the present disclosure, the agent is administered to the patient in an amount effective to decrease power in a theta band of the patient. In one aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the present disclosure, the decrease in power in the theta band of the patient causes a decrease in power in a gamma band frequency (greater than 30 Hz) of the patient.

In other aspects of the present disclosure, the agent is administered in an amount effective to moderate one or more conditions associated with thalamocortical dysrhythmia, cortical hyperexcitability marked by excessive or dysregulated gamma band activity, or thalamocortical dysrhythmia and cortical hyperexcitability marked by excessive or dysregulated gamma band activity. In an aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the present disclosure, the agent is administered in an amount effective to reduce theta band phase amplitude coupling with gamma band activity. In one aspect, the reduction in theta band phase amplitude coupling with gamma band activity reduces one or more symptoms associated with fragile X syndrome. Symptoms may include cognitive or intellectual disability, anxiety, receptive and expressive language delay, ADHD symptoms, insomnia, irritability marked by aggression, and self-injury. In another aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the present disclosure, the agent is administered in an amount effective to increase alpha band activity in the about 7 Hz to about 13 Hz frequency range to reduce one or more symptoms associated with fragile X syndrome. In yet another aspect of the present disclosure, the agent is administered in an amount effective to increase alpha band activity in the about 9 Hz to about 12.5 Hz frequency range. Symptoms associated with fragile X may include cognitive or intellectual disability, anxiety, receptive and expressive language delay, ADHD symptoms, insomnia, irritability marked by aggression, and self-injury. In one aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the present disclosure, the agent is administered in an amount effective to decrease the excitability of thalamic neurons to reduce one or more symptoms associated with fragile X syndrome. Symptoms may include cognitive or intellectual disability, anxiety, receptive and expressive language delay, ADHD symptoms, insomnia, irritability marked by aggression, and self-injury. In one aspect of the present disclosure, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect of the present disclosure, the agent is ethosuximide.

In another aspect of the present disclosure, the patient is administered a dose of 5-40 mg/kg of the agent per day. In one aspect, the agent is administered a dose of 500 mg per day. In yet another aspect of the present disclosure, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the disclosure, the patient is dosed flexibly to obtain a serum concentration range of 40-120 mcg/mL of the agent.

In another aspect of the present disclosure, the patient is administered the agent once per day, twice per day in equal doses, or multiple times per day in equal doses. In an aspect, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another aspect, the agent is ethosuximide.

In another aspect of the disclosure, the agent is administered to the patient orally in a capsule formulation or in a liquid formulation. In an example, the agent is a T-type calcium channel blocker, inhibitor, or antagonist selected from the group consisting of: mibefradil, ethosuximide, methsuximide, mesuximide, phensuximide, suvecaltamide, or one or more combinations thereof. In yet another example, the agent is ethosuximide.

Methods according to the present disclosure may comprise, consist of, or consist essentially of the elements of the methods as described herein, as well as any additional or optional element described herein or otherwise useful in the use of methods of treating fragile X syndrome or one or more symptoms associated with fragile X syndrome.

### BRIEF DESCRIPTION OF THE DRAWINGS

The foregoing and other features of the present disclosure will become more fully apparent from the following description, taken in conjunction with the accompanying drawings. Understanding that these drawings depict only several aspects in accordance with the disclosure and are, therefore, not to be considered limiting of its scope, the disclosure will be described with additional specificity and detail through use of the accompanying drawings. One of skill in the art may appreciate that certain features may be optional and that features or aspects of one example may be utilized or combined with other features highlighted in another example. Further, each and every aspect shown and described may not be necessary; rather, various aspects are shown and/or described as potential example features that may be included.

FIG. 1A illustrates an example model of thalamocortical dysrhythmia. The example model illustrates how changes in the thalamocortical system can cause abnormal electrical signals in the brain which are detectable on an EEG.

FIG. 1B illustrates an example of source-localized contrasts in key brain-wave frequency band powers in individuals with fragile X syndrome.

FIG. 1C provides an example graphical illustration of peak alpha frequency (measured in Hz) in individuals with fragile X syndrome compared to typically developing controls.

FIG. 1D provides an example graphical illustration of theta-gamma inverse coupling in males with fragile X syndrome theta, not alpha, is inversely coupled with gamma power in FXS.

FIG. 1E provides an example graphical illustration showing a correlation of increased gamma power and lower cognition measured using a non-verbal IQ test in males with fragile X syndrome.

FIG. 2A is an example model representing thalamocortical mechanisms leading to EEG readings in individuals with fragile X syndrome.

FIG. 2B is an example graphical representation of power spectral density plots showing that individuals with fragile X syndrome exhibit lower alpha power and increased theta power relative to controls of typically developing individuals.

### DETAILED DESCRIPTION

In this detailed description, reference is made to the accompanying drawings, which form a part hereof. In the drawings, similar symbols typically identify similar components, unless context dictates otherwise. The illustrative examples described in the detailed description, drawings, and potential points of novelty are not meant to be limiting. Other aspects may be utilized, and other changes may be made, without departing from the spirit or scope of the subject matter presented here. It will be readily understood that the aspects of the present disclosure, as described herein, and as examples are illustrated in the Figures, may be arranged, substituted, combined, and designed in a wide variety of different configurations, all of which are explicitly contemplated and make part of this disclosure.

Unless otherwise stated or context dictates otherwise, the use of the term “or” herein is the inclusive, and not the exclusive, use. For example, a list of “A, B, or C” includes either A, B, or C individually; A and B; B and C; A and C; or A, B, and C. Any percentages disclosed herein are percentages by weight unless otherwise indicated. Any reference to an amount of active ingredient refers to the active itself, notwithstanding the active being provided in a salt or other form.

As used herein, “an effective amount” refers to the amount of active agent required to confer the indicated therapeutic effect on the subject. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. Empirical considerations, such as the half-life, generally will contribute to the determination of the dosage. Although it is generally preferred that a maximum dose of the individual active agent or combinations thereof be used, that is, the highest safe dose according to sound medical judgment, in some cases it may be beneficial to administer less than the maximum safe dose. Frequency of administration may be determined and adjusted over the course of therapy, and is generally, but not necessarily, based on treatment or suppression or amelioration or delay of a target disease or disorder.

One aspect of the present disclosure is directed to treatments for patients with fragile X syndrome. Another aspect of the present disclosure is directed to methods for treating patients with fragile X syndrome that target one or more underlying causes of the symptoms associated with fragile X syndrome, which include, but are not limited to, cognitive or intellectual disability, anxiety, receptive and expressive language delay, ADHD symptoms, insomnia, irritability marked by aggression and self-injury.

One example aspect of the present disclosure includes orally administering an effective amount of ethosuximide to a patient with fragile X syndrome. Another aspect of the present disclosure includes orally administering an effective amount of a liquid formulation of ethosuximide to a patient with fragile X syndrome. Another aspect of the present disclosure includes orally administering an effective amount of a capsule formulation of ethosuximide to a patient with fragile X syndrome.

In one aspect of the disclosure, it was hypothesized that patients with fragile X syndrome may exhibit abnormal brain wave (electrical impulses generated by the brain) patterns. Brain waves are rhythmic or repetitive patterns of neural activity in the central nervous system. Neural tissue can generate oscillatory activity in many ways, either by mechanisms within individual neurons or by interactions between neurons. In individual neurons, oscillations can appear either as oscillations in membrane potential or as rhythmic patterns of action potentials, which then produce oscillatory activation of post-synaptic neurons. In neural ensembles, synchronized activity of large numbers of neurons can give rise to macroscopic oscillations, which can be observed in an electroencephalogram. Oscillatory activity in groups of neurons may arise from feedback connections between the neurons resulting in the synchronization of their firing patterns. EEGs may reveal oscillatory activity in specific frequency bands. For example, alpha activity may occur predominantly in the about 8 to about 12 Hz range. Theta activity may occur predominantly in the about 4 to about 8 Hz range. Gamma activity may occur predominantly in the about 30 to about 150 Hz range.

Patients with fragile X syndrome were monitored with EEG. EEG results were analyzed using source estimation and network analyses. It was found that patients with fragile X syndrome exhibited widespread circuit disruption and abnormalities in both low-frequency and high-frequency electrophysiologic activity and connectivity. The abnormalities in patients with fragile X syndrome are consistent with thalamocortical dysrhythmia.

Fragile X syndrome results in lost or disrupted FMRP protein function, which may affect the thalamocortical network. The loss of FMRP protein function in fragile X syndrome patients may lead to altered T-type calcium channel activity. The altered T-type calcium channel activity may contribute to thalamocortical dysrhythmia. However, unlike thalamocortical dysrhythmia in patients having absence seizures or epilepsy, thalamocortical dysrhythmia in fragile X syndrome patients exhibits in altered theta/alpha and gamma activity, which will be described in greater detail. This pattern of thalamocortical activity may underlie cognitive and sensory processing deficits in individuals with fragile X syndrome.

Thalamocortical dysrhythmia is an electrophysiological phenomenon exhibited is patients with certain neuropsychiatric conditions, including schizophrenia, epilepsy, Parkinson's disease, tinnitus, obsessive-compulsive disorder, neuropathic pain, and some instances of depression. Patients with thalamocortical dysrhythmia have abnormal electrical activity between the thalamus and the cerebral cortex.

Analyses of EEG readings of patients with fragile X syndrome have revealed a constellation of brain-wave abnormalities in fragile X syndrome patients consistent with thalamocortical dysrhythmia. Unlike patients experiencing acute symptoms such as seizures, where EEG measurements reveal 3 Hz spike wave discharges, brain wave abnormalities observed in patients with fragile X syndrome include (1) a reduced peak alpha frequency from the alpha range to the theta range; (2) a reduced alpha power with an anteriorized distribution; (3) an inversion of low-frequency coupling relationships with gamma power (i.e., theta-gamma couplings instead of alpha-gamma couplings); and (4) elevated gamma power.

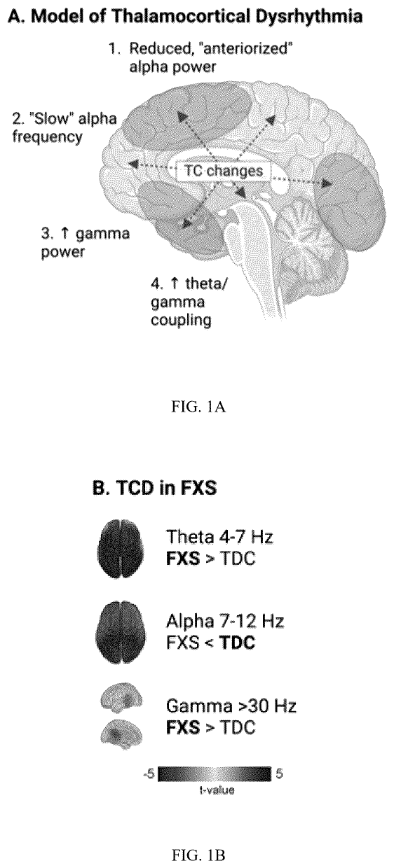

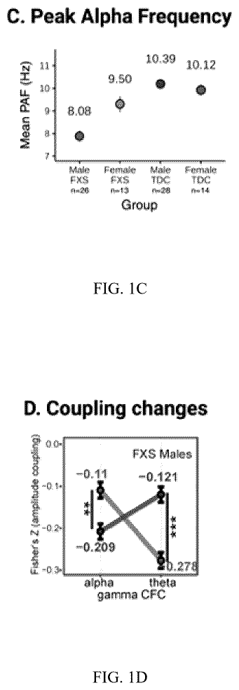

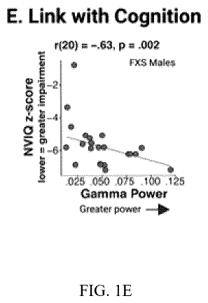

FIG. 1A illustrates an example model of thalamocortical dysrhythmia and shows a brain with thalamocortical dysrhythmia to illustrate the impact of thalamocortical dysrhythmia on cortical excitability in patients with fragile X syndrome. Thalamocortical dysrhythmia is a pattern of dysregulation of cortical excitability. The thalamus may have a modified role in contextual cortical modulation. Alpha oscillations may serve as a proxy for thalamocortical activity.

FIG. 1A provides an illustration of key changes observed in patient with thalamocortical dysrhythmia. In FIG. 1A, affected brain regions include the thalamus (approximately where the label “TC changes” appears), sensorimotor areas (shaded red), and frontal areas (shaded blue). FIG. 1A additionally lists certain changes, including reduced, anteriorized alpha power; slow alpha frequency; increased gamma power; and increased theta-gamma coupling.

In one aspect, patients with fragile X syndrome may experience a marked reduction in peak alpha frequency. Specifically, patients with fragile X syndrome may exhibit a reduction in peak alpha frequency from the higher alpha range (e.g., approximately 10 Hz) towards the theta range (e.g., approximately 6-7 Hz).

Peak alpha frequency is often correlated with individual differences in cognition. In typical development, peak alpha frequency increases from about 5 Hz theta in infancy to about 8 Hz low alpha in childhood to about 10 Hz or more high alpha in adolescents. In adulthood in a typically developing individual, peak alpha frequency is between about 10 Hz to about 13 Hz and may have special relevance to functional brain physiology. For example, neurons and oscillatory networks may demonstrate a frequency preference, like a radio receiver that can tune only specific electromagnetic frequencies. Thus, an “immature” peak alpha frequency (alone or along with reduced global alpha power) may be insufficient to drive neural ensembles with an alpha preference. Additionally, as theta dominance increases, alpha may lose its canonical role in top-down inhibition, which provide optimal timing windows for processing sensory and cognitive information.

In another aspect, alpha and gamma power abnormalities may be significantly associated with symptom severity in patients with fragile X syndrome, including cognitive function, anxiety, social communication, and sensory sensitivities.

FIG. 1B illustrates an example of source-localized contrasts in key brain-wave frequency band powers in individuals with fragile X syndrome. As seen in FIG. 1B, in patients with fragile X syndrome compared to typically developing controls (“TDC”), fragile X syndrome patients may exhibit (a) increased theta band power in the 4-7 Hz frequency range, (b) decreased alpha band power in the 7-12 Hz frequency range, and/or increased gamma band power in the frequency range greater than 30 Hz.

FIG. 1C provides an example graphical illustration of peak alpha frequency (measured in Hz) in individuals with fragile X syndrome compared to typically developing controls. Typically developing males may exhibit a peak alpha frequency of 10.39 Hz, and typically developing females may exhibit a peak alpha frequency of about 10.12 Hz. Males with fragile X syndrome may exhibit a peak alpha frequency of about 8.08 Hz, females with fragile X syndrome may exhibit a peak alpha frequency of about 9.50 Hz. It is known that females with fragile X syndrome typically have less severe symptoms than males, and the data indicates a peak alpha frequency of females with fragile X syndrome closer to normal than males with fragile X syndrome. FIG. 1C may support a correlation between the amount of peak alpha frequency decrease and symptom severity in individuals with fragile X syndrome.

FIG. 1D provides a graphical illustration of theta-gamma inverse coupling in males with fragile X syndrome. In typically developing individuals, it is expected that alpha frequences are inversely coupled with gamma power, i.e., that the amplitude of the gamma brain waves is inversely related to the phase of alpha brain waves, indicating that the alpha phase modulates the activity of gamma oscillations. FIG. 1D shows that theta (not alpha) frequencies are inversely coupled with gamma power in males with fragile X syndrome. Thus, FIG. 1D may support that in individuals with fragile X syndrome, theta phase brain waves modulates the activity of gamma oscillations.

FIG. 1E provides an example graphical illustration showing a correlation of increased gamma power and lower cognition measured using a non-verbal IQ test in males with fragile X syndrome. FIG. 1E may support that, as gamma power increases in an individual, IQ tends to decrease, and thus may support the notion that increases in gamma power observed in individuals with fragile X syndrome may result in lower IQ, which is a recognized symptom of fragile X syndrome.

One aspect of the present disclosure includes methods of treating a patient having fragile X syndrome with an effective amount of an agent that modulates activity of a T-type calcium channel. The agent can be a T-type calcium channel blocker, inhibitor, or antagonist, and more specifically be a T-type calcium channel blocker, inhibitor, or antagonist selected from the group consisting of: mibefradil, ethosuximide, methsuximide, mesuximide, phensuximide, suvecaltamide, or one or more combinations thereof. In one specific example, the present disclosure includes orally administering an effective amount of ethosuximide to a patient with fragile X syndrome. Another aspect of the present disclosure includes orally administering an effective amount of a liquid formulation of ethosuximide to a patient with fragile X syndrome. In another aspect, the present disclosure includes orally administering an effective amount of a capsule formulation of ethosuximide to a patient with fragile X syndrome.

Ethosuximide may affect neuronal excitability. Ethosuximide may be known as a T-type voltage sensitive calcium channel modulator. In one aspect, ethosuximide may block T-type calcium channels. In other aspects, ethosuximide may have effects on other classes of ion channels. Ethosuximide may block all T-type calcium channel isoforms and is considered an antagonist of T-type calcium channels. Significant T-type calcium channel density occurs in dendrites of neurons. Thus, ethosuximide may exhibit pronounced effects in the dendrites of neurons.

One aspect of the present disclosure may include using one or more acute pharmacological probes to evaluate whether an abnormal circuit mechanism like thalamocortical dysrhythmia underlies the observed neural activity alterations in individuals with fragile X syndrome. In an aspect, an effective amount of a dose of an agent that modulates activity of a T-type calcium channel, or more specifically a T-type calcium channel blocker, inhibitor, or antagonist selected from the group consisting of mibefradil, ethosuximide; methsuximide, mesuximide, phensuximide, suvecaltamide, or one or more combinations thereof may be administered to a patient with fragile X syndrome. A T-type calcium channel blocker may be associated with a reduction in mean spectral density in the theta band range (about 2.5 Hz to about 6.5 Hz) in patients with enhanced baseline theta activity. As fragile X syndrome patients may exhibit an enhanced baseline theta activity, the T-type calcium channel blocker, inhibitor, or antagonist selected from mibefradil, ethosuximide, methsuximide, mesuximide, phensuximide, suvecaltamide, or one or more combinations thereof may be expected to reduce the theta band activity, which may lead to a decrease in symptom severity in fragile X syndrome patients.

In another aspect of the present disclosure, ethosuximide may be associated with a reduction in theta band phase amplitude coupling with high-frequency (e.g., gamma) band activity. Ethosuximide is a front-line treatment for absence seizures, and absence seizures may be associated with an increased theta band phase amplitude coupling with high-frequency (e.g., gamma) band activity. Administration of an effective amount of a T-type calcium channel blocker, inhibitor, or antagonist selected from mibefradil, ethosuximide, methsuximide, mesuximide, phensuximide, suvecaltamide, or one or more combinations thereof to fragile X syndrome patients may lead to a reduction of theta band phase amplitude coupling with high-frequency (e.g., gamma) band activity, which may in turn lead to an improvement of symptoms associated with fragile X syndrome.

In another aspect of the present disclosure, a T-type calcium channel blocker, inhibitor, or antagonist selected from mibefradil, ethosuximide, methsuximide, mesuximide, phensuximide, suvecaltamide, or one or more combinations thereof may be associated with an increase in alpha-band activity in the about 7 Hz to about 13 Hz frequency range. Administration of an effective amount of the T-type calcium channel blocker, inhibitor, or antagonist selected from mibefradil, ethosuximide, methsuximide, mesuximide, phensuximide, suvecaltamide, or one or more combinations thereof to fragile X syndrome patients may lead to an increase in alpha-band activity in the about 10 Hz to about 13 Hz range, which may in turn lead to an improvement of symptoms associated with fragile X syndrome.

In another aspect of the present disclosure, a T-type calcium channel blocker, inhibitor, or antagonist selected from mibefradil, ethosuximide, methsuximide, mesuximide, phensuximide, suvecaltamide, or one or more combinations thereof may regulate T-type calcium channels by blocking or inhibiting their activity, which may reduce the flow of calcium ions into neurons during calcium spikes, which may decrease the excitability of thalamic neurons and disrupt one or more abnormal rhythmic neuron firing patterns associated with thalamocortical dysrhythmia and fragile X syndrome patients.

In another aspect of the present disclosure, instead of or in addition to ethosuximide, a T-type calcium channel blocker, inhibitor, or antagonist may be administered to a fragile X syndrome patient. In another aspect of the present disclosure, instead of or in addition to ethosuximide, mibefradil, methsuximide, mesuximide, or phensuximide, or one or more combinations thereof, may be administered to a fragile X syndrome patient.

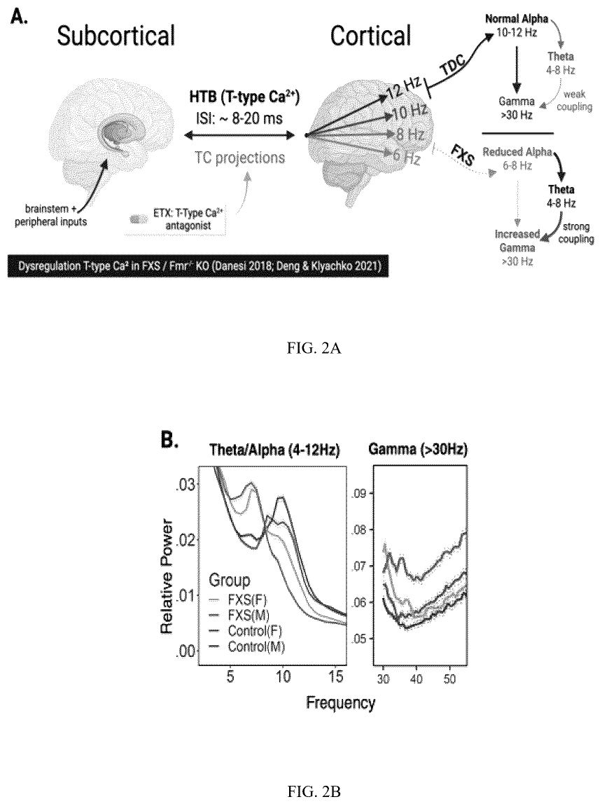

FIG. 2A represents a model of thalamocortical mechanisms leading to fragile-X-syndrome-associated EEG findings. In the subcortical portion of the model, thalamic neurons may receive inputs from brainstem and peripheral sources. Thalamocortical (TC) projections may indicate high-threshold bursts (HTB) mediated by T-type calcium (Ca²⁺) channels with inter-spike intervals (ISi) of ~8-20 ms that may drive frequency of cortical oscillations. In the cortical portion of the model, TC projections may regulate low-frequency activity (5 Hz to 12 Hz). In individuals with fragile X syndrome, reduced alpha frequency (6-8 Hz) (represented in FIG. 2A with a dashed red arrow) may lead to enhanced theta-gamma coupling and increased gamma activity (>30 Hz). Ethosuximide blocks T-type calcium (Ca²⁺) channels in thalamic neurons, which may increase the alpha peak frequency and normalize thalamocortical oscillations. Administration of ethosuximide may regulate the thalamocortical dysrhythmia phenomena observed in fragile X syndrome patients, which may improve symptom severity in these patients. Instead of or in addition to ethosuximide, a T-type calcium channel blocker, inhibitor, or antagonist selected from mibefradil, ethosuximide, methsuximide, mesuximide, phensuximide, suvecaltamide, or one or more combinations thereof may be administered to a fragile X syndrome patient, which may help regulate the thalamocortical dysrhythmia phenomena observed in fragile X patients and lead to improved symptom severity. Instead of or in addition to ethosuximide, a T-type calcium channel blocker, inhibitor, or antagonist selected from mibefradil, ethosuximide, methsuximide, mesuximide, phensuximide, suvecaltamide, or one or more combinations thereof may be administered to a fragile X syndrome patient, which may help: (a) moderate one or more conditions associated with thalamocortical dysrhythmia; (b) raise the peak alpha frequency of the patient; (c) decrease power in the theta band of the patient; (d) reduce theta band phase amplitude coupling with gamma band activity; (e) increase alpha band activity in the about 10 Hz to about 13 Hz frequency range; (f) decrease the excitability of thalamic neurons; (g) decrease the power in the gamma band frequency (greater than 30 Hz); or combinations thereof.

FIG. 2B is an example graphical representation of power spectral density plots showing that individuals with fragile X syndrome exhibit lower alpha power and increased theta power relative to controls of typically developing individuals. FIG. 2B shows alpha oscillation variability by genotype (fragile X syndrome versus typical control) and sex (male versus female). FIG. 2B shows power spectral density plots in fragile X syndrome individuals and typically developing controls, separated by sex (M: male, F: female). Individuals with fragile X syndrome (represented by red and orange) exhibit lower alpha power and increased theta power compared to typically developing controls (represented by blue and purple), with notable sex-specific differences in both groups.

In an aspect of the present disclosure, a patient having fragile X syndrome may be administered about 5-40 mg/kg of the agent daily. In another aspect, a patient having fragile X syndrome may be administered 5-40 mg/kg of the agent per day. In specific aspects, the agent is ethosuximide. The agent may be administered once per day or multiple times per day (e.g., two, three, or four times per day) in equal or unequal doses. In another aspect, the patient is dosed flexibly to obtain a serum concentration range of 40-120 mcg/mL of the agent, for example ethosuximide, with dosing from 5-40 mg/kg per day. The agent may be administered as an oral capsule formulation. The agent may alternatively be administered in an oral liquid formulation. A dosage amount of 5-40 mg/kg per day, or 500 mg per day for a pediatric patient, may be effective to treat one or more symptoms of fragile X syndrome. A dosage amount of 5-40 mg/kg per day, or for example 500 mg per day for a pediatric patient, may be effective to cause one or more of the following effects in a fragile X syndrome patient: an increase in peak alpha frequency, a decrease of power in the theta band, a decrease in power in the gamma band, a reduction in thalamocortical dysrhythmia or one or more effects of thalamocortical dysrhythmia, a decrease in the excitability of thalamic neurons, or combinations thereof.

In an aspect of the present disclosure, administration of a T-type calcium channel blocker, inhibitor, or antagonist selected from mibefradil, ethosuximide, methsuximide, mesuximide, phensuximide, suvecaltamide, or one or more combinations thereof may contribute to a measurable improvement in one or more phenotypic functions in patients with fragile X syndrome. For example, a measurable improvement in adaptive behavior as measured by the Vineland Adaptive Behavior Scale, cognition as measured by the NIH toolbox, global clinical improvement as measured by the Clinical Global Impressions Improvement Subscale CGI-I, or combinations thereof, may be realized.

The specific examples described herein are exemplary only and are not limiting to the aspects of the disclosure defined by the claims. All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. It is, therefore, to be understood that the foregoing examples are presented by way of example only and that, within the scope of the appended claims and equivalents thereto, inventive aspects may be practiced otherwise than as specifically described, and the disclosure should be construed as encompassing those aspects.

What is claimed is:

1. A method of treating fragile X syndrome, comprising administering to a patient having fragile X syndrome an effective amount of an agent that modulates activity of a T-type calcium channel.

2. A method of treating one or more symptoms associated with fragile X syndrome, comprising administering to a patient having fragile X syndrome an effective amount of an agent that modulates activity of a T-type calcium channel.

3. The method of claim 2, wherein the agent is a T-type calcium channel blocker, inhibitor, or antagonist selected from the group consisting of: mibefradil, ethosuximide, methsuximide, mesuximide, phensuximide, suvecaltamide, or one or more combinations thereof.

4. The method of claim 2, wherein the agent is administered in an amount effective to raise a peak alpha frequency of the patient.

5. The method of claim 4, wherein the agent is a T-type calcium channel blocker, inhibitor, or antagonist selected from the group consisting of: mibefradil, ethosuximide, methsuximide, mesuximide, phensuximide, suvecaltamide, or one or more combinations thereof.

6. The method of claim 2, wherein the agent is administered to the patient in an amount effective to decrease power in a theta band of the patient.

7. The method of claim 6, wherein the agent is a T-type calcium channel blocker, inhibitor, or antagonist selected from the group consisting of: mibefradil, ethosuximide, methsuximide, mesuximide, phensuximide, suvecaltamide, or one or more combinations thereof.

8. The method according to claim 4, wherein the rise in peak alpha frequency causes a decrease in power in a gamma band frequency (greater than 30 Hz) of the patient.

9. The method according to claim 6, wherein the decrease in power in the theta band of the patient causes a decrease in power in a gamma band frequency (greater than 30 Hz) of the patient.

10. The method of claim 2, wherein the agent is administered in an amount effective to moderate one or more conditions associated with thalamocortical dysrhythmia, cortical hyperexcitability marked by excessive or dysregulated gamma band activity, or thalamocortical dysrhythmia and cortical hyperexcitability marked by excessive or dysregulated gamma band activity.

11. The method of claim 2, wherein the agent is administered in an amount effective to reduce theta band phase amplitude coupling with gamma band activity.

12. The method of claim 11, wherein the reduction in theta band phase amplitude coupling with gamma band activity reduces one or more symptoms associated with fragile X syndrome.

13. The method of claim 2, wherein the agent is administered in an amount effective to increase alpha band activity in the about 7 Hz to about 13 Hz frequency range to reduce one or more symptoms associated with fragile X syndrome.

14. The method of claim 2, wherein the agent is administered in an amount effective to decrease the excitability of thalamic neurons to reduce one or more symptoms associated with fragile X syndrome.

15. The method of claim 2, wherein the patient is administered a dose of 5-40 mg/kg of the agent per day.

16. The method of claim 2, wherein the patient is administered a dose of 500 mg of the agent per day.

17. The method of claim 15, wherein the agent is administered to the patient once per day, twice per day in equal doses, multiple times per day in equal doses.

18. The method of claim 15, wherein the patient is dosed flexibly to obtain a serum concentration range of 40-120 mcg/mL of the agent.

19. The method of claim 15, wherein the agent is administered to the patient orally in a capsule formulation.

20. The method of claim 15, wherein the agent is administered to the patient orally in a liquid formulation.
